(19) United States
(12) Patent Application Publication
(10) Pub. No.: US 2009/0305421 A1
(43) Pub. Date: Dec. 10, 2009

(54) Recombinant vector for deleting specific regions of chromosome and method for deleting specific chromosomal regions of chromosome in the microorganism using the same

(76) Inventors: Sun Chang Kim, Daejeon (KR); Kui Hyeon Kang, Daejeon (KR); Byung Jo Yu, Daejeon (KR); Jun Hyoung Lee, Daejeon (KR); Bong Hyun Sung, Daejeon (KR); Choong Hoon Lee, Daejeon (KR); Sang Hee Lee, Daejeon (KR); Ju Young Lee, Daejeon (KR); Myung Keun Park, Daejeon (KR)

Correspondence Address: CHRISTIE, PARKER & HALE, LLP, PO BOX 7068, PASADENA, CA, 91109-7068, US

(21) Appl. No.: 12/291,007

(22) Filed: Nov. 4, 2008

(30) Foreign Application Priority Data
Feb. 11, 2008 (KR) 10-2008-0012377

(51) Int. Cl. C12N 15/70; C12N 1/21

(52) U.S. Cl. 435/471; 435/320.1; 435/252.33

(57) ABSTRACT

Disclosed herein are a recombinant vector for deletion of specific chromosomal regions and a method for deletion of targeted microbial chromosomal regions using the same. Specifically, the recombinant vector comprises an arabinose-inducible promoter; a gene encoding a protein involved in lambda (λ)-red recombination; a rhamnose-inducible promoter; and a gene encoding the I-SceI endonuclease. The present invention enables a convenient, rapid and markerless successive deletion of specific genes of microbes, as compared to a conventional method.

### CROSS-REFERENCE TO RELATED APPLICATION

This application claims priority to and the benefit of Korean Patent Application Number 10-2008-0012377, filed on Feb. 11, 2008, in the Korean Intellectual Property Office, the entire content of which is incorporated herein by reference.

### BACKGROUND OF THE INVENTION

1. Field of the Invention

The present invention relates to a recombinant vector for deletion of specific chromosomal regions, which is capable of providing efficient and easy simultaneous deletion of specific chromosomal regions of a target microbe and a method for deletion of targeted microbial chromosomal regions using the same.

2. Description of the Related Art

Striking development of biotechnologies opened the era of post-genomics. Keeping pace with current trends, industrial strains that can be widely and beneficially used in biotechnology industry have been produced taking advantage of genetic information of diverse organisms. However, a large majority of currently available industrial strains have problems such as excessive energy consumption and by-product production by gene clusters producing useless materials and therefore are not favorable for industrial-scale production of high-purity useful materials. To this end, a great deal of research has been focused on development of techniques which are capable of accomplishing rapid, efficient and convenient deletion of industrially useless gene clusters from genomes of target microbes, for artificial construction of novel high-functionality metabolically engineered strains and cell lines with remarkably enhanced productivity through the deletion of useless gene clusters from the selected microbial genomes.

Conventional known techniques for deletion of specific regions of Escherichia coli (E. coli) chromosomes involve use of a linear DNA fragment containing a recombination region of a specific region to be deleted and a selectable marker, in conjunction with two different vectors having functions necessary for genomic insertion and deletion of the linear DNA fragment. Specifically, in a first step of the conventional gene deletion strategy, a targeted chromosomal region is deleted through recombinational insertion of a linear DNA fragment having a selectable marker into a specific chromosomal region to be deleted, using a vector having recombination functions. This is followed by removal of the introduced vector from E. coli. In a second step, a vector intended for removal of the selectable marker of the linear DNA fragment which was used in deletion of the specific chromosomal region is introduced into the E. coli host to thereby remove the selectable marker gene. Therefore, when it is desired to delete multiple specific chromosomal regions of E. coli using the conventional gene deletion methodology, it is disadvantageous in that the conventional technique requires sequential introduction and subsequent removal of two vectors necessary for each deletion step, thus resulting in complicated and time-consuming processes.

### SUMMARY OF THE INVENTION

Therefore, the present invention has been made in view of the above problems, and it is an object of the present invention to provide a recombinant vector for deletion of specific gene regions, which is capable of achieving rapid, efficient and successive deletion of specific gene regions of a microbe, and a method for deletion of specific microbial chromosomal regions using the same.

In accordance with an aspect of the present invention, the above and other objects can be accomplished by the provision of a recombinant vector for deletion of specific chromosomal regions, comprising an arabinose-inducible promoter (Pₐᵣₐ); a gene encoding a protein involved in lambda (λ)-red recombination; a rhamnose-inducible promoter (Pᵣₕₐ); and a gene encoding the I-SceI endonuclease, wherein the vector has a base sequence of SEQ ID NO: 1 and is represented by a cleavage map of FIG. 1.

In accordance with another aspect of the present invention, there is provided Escherichia coli transformed with the aforesaid recombination vector.

In accordance with yet another aspect of the present invention, there is provided a method for deletion of specific chromosomal regions of a microbe using the aforesaid recombination vector, comprising the steps of:

1) preparing a linear DNA fragment containing homology arms A and B which are involved in λ-red recombination when they are introduced into a target microbe; a selectable marker; an I-SceI recognition site which is involved in homologous recombination for removal of the selectable marker; and a homology arm C which is involved in homologous recombination for removal of the selectable marker;

2) introducing the linear DNA fragment into a microbe transformed with the aforesaid recombination vector to replace a specific locus of the microbial chromosome with the linear DNA fragment through λ-red recombination between the homology arms of the DNA fragment and the microbial chromosome regions homologous to the homology arms; and

3) culturing the specific chromosomal locus-replaced microbe in a rhamnose-containing medium to induce expression of the I-SceI endonuclease, such that homologous recombination between the homology arm C of the DNA fragment and the microbial chromosomal region homologous to the homology arm C is driven to remove the selectable marker,

wherein the homology arm A is a region homologous to 50 to 500 bp of one end of the deletion target domain of a microbial chromosome and the homology arm B is a region homologous to 50 to 500 bp of the other end of the deletion target domain of the microbial chromosome, and

the homology arm C is a region that is homologous to a 300-500 bp region contiguous to either one of the microbial chromosome regions homologous to the homology arm A and homology arm B.

In one embodiment of the present invention, the method may include repeating Steps 1 and 2 to prepare a plurality of different linear DNA fragments and introducing the linear DNA fragments into a microbe transformed with a recombination vector of claim 1 to delete a plurality of specific microbial chromosomal regions.

In one embodiment of the present invention, the selectable marker may be at least one selected from the group consisting of a chloramphenicol-resistant gene having a base sequence of SEQ ID NO: 13, a kanamycin-resistant gene having a base sequence of SEQ ID NO: 14, and sacB.

In one embodiment of the present invention, the method may further comprise culturing the microbes in a sucrose-containing medium after the step of removing the selectable marker.

In one embodiment of the present invention, the homology arm A, the homology arm B and the homology arm C may have base sequences of SEQ ID NO: 15, SEQ ID NO: 16 and SEQ ID NO: 17, respectively. In another embodiment of the present invention, the homology arm A, the homology arm B and the homology arm C may have base sequences of SEQ ID NO: 22, SEQ ID NO: 23 and SEQ ID NO: 24, respectively. In yet another embodiment of the present invention, the homology arm A, the homology arm B and homology arm C may have base sequences of SEQ ID NO: 31, SEQ ID NO: 32 and SEQ ID NO: 33, respectively.

In one embodiment of the present invention, the specific chromosomal region of the microbe may contain a gene essential for survival of the microbe, and the linear DNA fragment may further contain the aforesaid essential survival gene between the homology arm A and the homology arm C.

In one embodiment of the present invention, the essential gene is argS having a base sequence of SEQ ID NO: 43, and the homology arm A, the homology arm B and the homology arm C may have base sequences of SEQ ID NO: 40, SEQ ID NO: 41 and SEQ ID NO: 42, respectively.

### BRIEF DESCRIPTION OF THE DRAWINGS

FIG. 1 is a cleavage map of a recombinant vector pREDI for deletion of chromosomes in accordance with the present invention;

FIG. 2 is a schematic diagram illustrating deletion of a selectable marker, using a recombination vector pREDI in accordance with the present invention;

FIG. 3a is a diagram showing insertion of two linear DNA fragments containing different selectable markers into two specific genomic regions of E. coli and simultaneous deletion of the inserted linear DNA fragments, using a recombination vector pREDI in accordance with the present invention;

FIG. 3b is a photograph showing electrophoretic patterns for the results of PCR amplification conducted to confirm the insertion of two linear DNA fragments and the degree of genomic deletion after insertion and deletion of two linear DNA fragments into and from two specific genomic regions of E. coli (M: marker, Lane 1: MG1655 strain not transformed with a linear DNA fragment (positive control), Lane 2: PCR results confirming that a DNA fragment A-C-Cm sacB I-SecI-B, constructed to delete a b0980-b1052 region, was successfully introduced into the E. coli strain, Lane 3: PCR results confirming that a DNA fragment A-C-Km sacB I-SecI-B, constructed to delete a b1137-b1168 region, was successfully introduced into the E. coli strain, Lane 4: PCR results confirming that a DNA fragment A-C-Cm sacB I-SecI-B, constructed to delete a b0980-b1052 region, was successfully introduced into the E. coli strain, and a Cm sacB I-SecI-B portion was then removed by the action of I-SecI, Lane 5: PCR results confirming that a DNA fragment A-C-Km sacB I-SecI-B, constructed to delete a b1137-b1168 region, was successfully introduced into the E. coli strain, and a Km sacB I-SecI-B portion was then deleted by the action of I-SecI, and Lanes 6 and 7: PCR results for each region between b0980-b1052 and between b1137-b1168, conducted to confirm whether b0980-b1052 and b1137-b1168 were successfully deleted from the E. coli strain);

FIG. 4a is a diagram illustrating deletion of a specific genomic region containing an essential survival gene, in deletion of specific genomic regions using a recombination vector pREDI in accordance with the present invention; and

FIG. 4b is a photograph showing electrophoretic patterns of the results of PCR amplification conducted to confirm the insertion of a linear DNA fragment and the degree of genomic deletion, after deletion of a specific genomic region containing an essential survival gene (Lane 1: MG1655 strain not transformed with linear DNA fragment, Lane 2: MG1655 strain transformed with a linear DNA fragment, Lane 3: MG1655 strain transformed with a linear DNA fragment, Lane 4: MG1655 strain with no deletion of chromosome, and Lane 5: MG1655 strain with deletion of a specific chromosomal region).

### DETAILED DESCRIPTION OF THE PREFERRED EMBODIMENTS

As a result of a variety of extensive and intensive studies and experiments to solve the problems as described above, i.e. disadvantages of a conventional art requiring the use of two vectors for λ-red recombination and I-SceI endonuclease expression functions which are employed in deletion of specific chromosomal regions of microorganisms, the inventors of the present invention succeeded in construction of a recombination vector pREDI which enables expression of the above-mentioned two functions by a single vector using different induction methods. Therefore, the present invention relates to a recombinant vector pREDI for deletion of specific chromosomal regions of a microbe, E. coli transformed with the same recombination vector pREDI, and a method for deletion of specific chromosomal regions of a microbe, using a linear DNA fragment containing the recombination vector pREDI and a selectable marker.

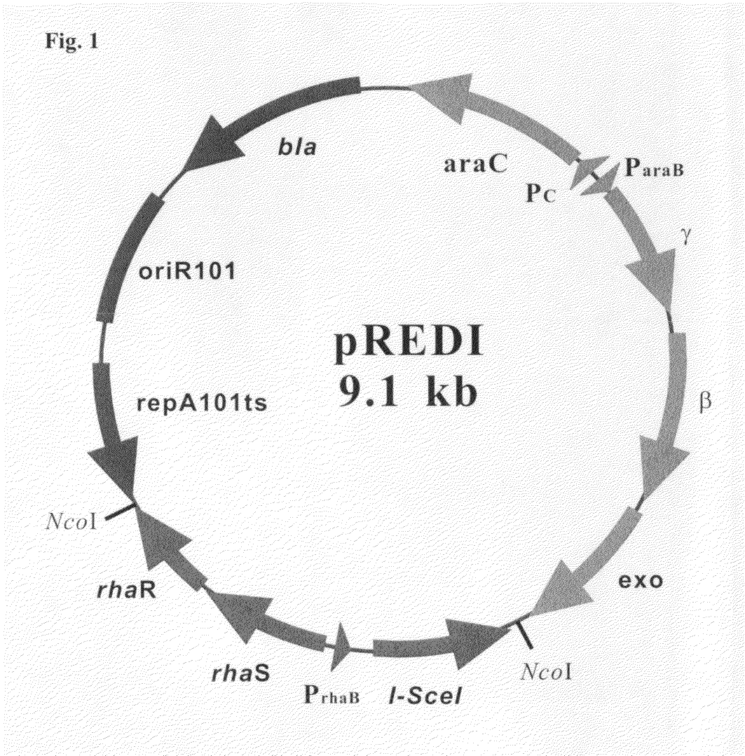

More specifically, the present invention provides a recombinant vector for deleting specific chromosomal regions of a microbe, comprising an arabinose-inducible promoter (Pₐᵣₐ); a gene encoding a protein involved in lambda (λ)-red recombination; a rhamnose-inducible promoter (Pᵣₕₐ); and a gene encoding the I-SceI endonuclease, wherein the recombinant vector has a base sequence of SEQ ID NO: 1 and is represented by a cleavage map of FIG. 1.

In the context of the present invention, the protein involved in λ-red recombination is a conjugated protein consisting of Gam, Bet and Exo, as disclosed in the literature including Datsenko, K. A. et al., each of which is expressed by γ, β and exo genes. Gam inhibits the host RecBCD exonuclease V so that Bet and Exo can gain access to DNA ends to promote recombination (Datsenko, K. A. et al., Proc. Natl. Acad. Sci. 97:6640, 2000; Murphy, K. C., J. Bacteriol., 180:2063, 1998).

For expression of the protein involved in arabinose-inducible λ-red recombination in the context of the present invention, the aforesaid recombination vector contains a gene encoding the λ-red recombination protein, e.g. γ/β/exo gene (SEQ ID NO: 3), in conjunction with the arabinose-inducible promoter (Pₐᵣₐ) (SEQ ID NO: 2). Further, the λ-red recombination protein (γ, β, exo) allows the occurrence of λ-red recombination upon homologous recombination-mediated insertion of the linear DNA fragment into a target chromosome of interest. A gene encoding Gam, a gene encoding Bet and a gene encoding Exo may have base sequences of SEQ ID NO: 4, SEQ ID NO: 5 and SEQ ID NO: 6, respectively.

Further, the recombination vector in accordance with the present invention is configured to exert rhamnose-inducible I-SceI expression which consequently results in specific cleavage of the I-SceI recognition site. According to such a configuration, the thus-expressed I-SceI endonuclease cleaves the I-SceI recognition site contained in the linear DNA fragment incorporated into the genome to thereby facilitate homologous recombination between the partial chromosomal fragment contained in the linear DNA and the microbial chromosomal region homologous to that chromosomal fragment. In order to achieve rhamnose-inducible I-SceI expression, the recombination vector of the present invention may comprise the rhamnose-inducible promoter (Pᵣₕₐ) (SEQ ID NO: 7) and a gene encoding the endonuclease I-SceI (SEQ ID NO: 8).

Further, the present invention relates to E. coli transformed with the aforesaid recombination vector.

In the context of the present invention, the method of deleting specific chromosomal regions of a microbe via the use of a linear DNA fragment containing the recombination vector pREDI and the selectable marker specifically includes the following steps:

(1) preparing a linear DNA fragment containing a selectable marker, a sacB gene, an I-SceI recognition site and homology arms that are partially homologous to a microbial chromosome;

(2) transforming the pREDI-transformed microbe with the aforesaid linear DNA fragment to thereby replace the target chromosomal region of the microbe with the linear DNA fragment of Step 1 by a λ-red recombination protein that is expressed under the control of an arabinose-inducible promoter in the recombination vector pREDI; and

(3) eliminating the remaining selectable marker from the microbial genome, by expression of the I-SceI endonuclease that is under the control of a rhamnose-inducible promoter of the recombination vector pREDI harboring in the DNA fragment-transformed microbe.

Further, the above deletion method may further comprise Step 4 of simultaneously deleting two or more genomic regions of the microbe through insertion of two or more linear DNA fragments containing different selectable markers.

In addition, the above method may comprise an additional step 5 of deleting a specific region containing a gene essential for survival of a microbe. Therefore, the genomic deletion method of the present invention enables efficient deletion of only the desired specific genomic region within a short period of time while retaining the essential gene, using a linear DNA fragment containing the essential gene.

In Step 1 of the method of deleting a specific microbial chromosomal region using the recombinant vector of the present invention for deletion of specific chromosomal regions, the linear DNA fragment contains a selectable marker; 50-500 bp homology arms A and B involved in λ-red recombination and homologous to a portion of a microbial chromosome; an I-SceI recognition site necessary for the removal of the selectable marker; a 300-500 bp homology arm C; and a sacB gene to confirm the markerless deletion of the target regions.

Examples of the selectable marker that can be used in the present invention may include, but are not limited to, a chloramphenicol-resistant gene (Cm^{R}), a kanamycin-resistant gene (Km^{R}), and tetracycline-resistant gene (Tc^{R}). A mutant strain harboring a chromosome which is recombined with a DNA fragment containing such a selectable marker gene acquires resistance to the corresponding antibiotic, which enables selection of the mutant strain devoid of a specific chromosomal region.

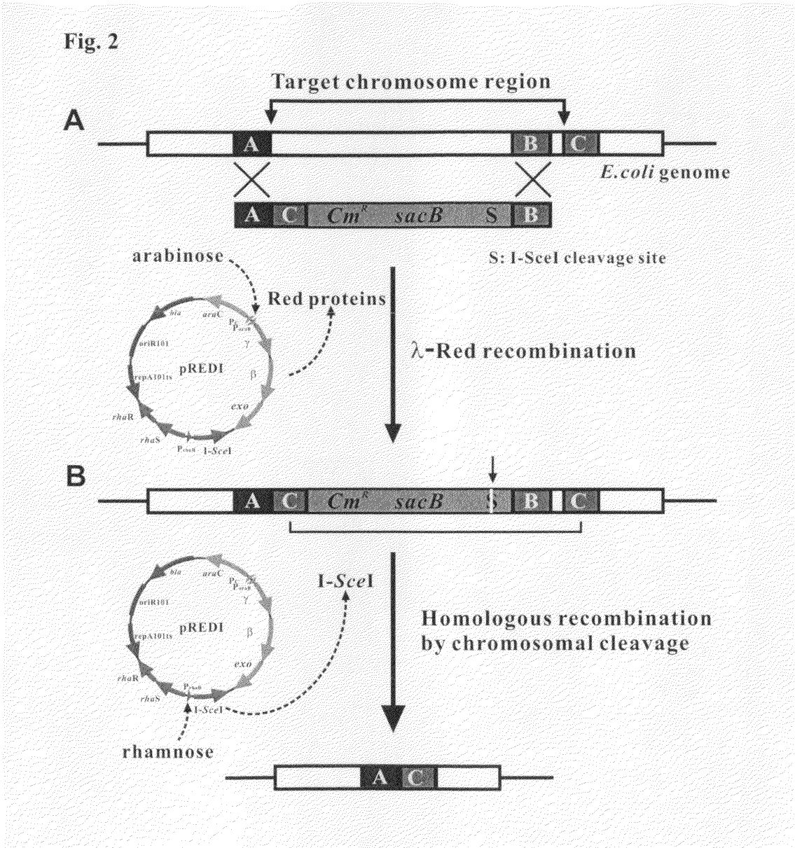

As shown in FIG. 2, the homology arm refers to a region homologous to a microbial chromosome which is sought to be modified or deleted, and is designated as homology arms A, B and C, respectively, in sequence lists and drawings. Homology arms A and B are obtained by PCR amplification of 50-500 bp flanking sequences on both ends of the microbial chromosome to be deleted. They are positioned at respective opposite ends of the linear DNA fragment and participate in λ-red recombination for introduction of the linear DNA fragment into the microbe. The homology arm C is obtained by PCR amplification of a 50-500 bp flanking sequence on either end of the microbial chromosome homologous to the homology arm A or B, is positioned next to a homologous region on either end of the linear DNA fragment, and is involved in the homologous recombination for removal of the selectable marker. Herein, the homology arms A, B and C may vary depending on kinds of target microorganisms and genomic regions to be deleted. In one embodiment of the present invention, each of the homology arms A, B and C may have a base sequence of SEQ ID NO: 15, 16 and 17, SEQ ID NO: 22, 23 and 24, SEQ ID NO: 31, 32 and 33, or SEQ ID NO: 40, 41 and 42, even though they are not limited thereto.

The I-SceI recognition site (SEQ ID NO: 52) is 18 bp in length. Since the E. coli chromosome contains no I-SceI recognition site, the linear DNA fragment used for deletion of a specific gene must contain the I-SceI recognition site.

In Step 2, a microbial strain is transformed with a recombination vector by a conventional electroporation technique, and the linear DNA fragment is then electroporated into the microbial strain. As shown in FIG. 2, a targeted chromosomal site is deleted by replacement of a specific site of the microbial strain chromosome with the DNA fragment, through λ-red recombination between the homology arms A and B located respectively on opposite ends of the linear DNA fragment and homologous to the microbial chromosome and the corresponding homologous microbial chromosome region. Then, the microbial mutants having a chromosomal replacement with the linear DNA fragment are cultured and selected in a medium containing the corresponding antibiotic depending on the kinds of selectable markers. Further, the λ-red recombination-mediated gene replacement in the thus-selected mutants can be confirmed by PCR.

In Step 3, as shown in FIG. 2, the microbial mutants replaced with the linear DNA fragment are selected and cultured to allow rhamnose-inducible I-SceI expression, which, in turn, results in promotion of the homologous recombination between the homology arm C positioned at one end of the linear DNA fragment and the microbial chromosome region homologous to the homology arm C, thus leading to deletion of the selectable marker from the microbial mutants. Since transcription of an I-SceI gene in the recombination vector is under the control of a rhamnose-inducible promoter, mutants harboring the recombination vector introduced therein are cultured in a medium supplemented with rhamnose to induce expression of the I-SceI gene, which consequently results in site-specific cleavage of the I-SceI recognition site. In this manner, specific cleavage of the I-SceI recognition site facilitates homologous recombination between the region of the linear DNA fragment having homology with a microbial chromosome portion and the corresponding homologous chromosomal region of the microbe.

Further, the microbial mutants are cultured in a sucrose-containing medium to thereby express a sacB gene harbored in the linear DNA fragment introduced into the microbial mutants, thus causing toxicity to cells. Generally, the sacB gene encodes exoenzyme levansucrase which catalyzes degradation of sucrose into glucose and fructose and synthesis of levan, a polymer of fructose. Because the levan is toxic to cells, microorganisms having the sacB gene (SEQ ID NO: 53) cannot survive in a sucrose-containing medium. Therefore, the microbial mutants having homologous recombination-mediated deletion of the selectable marker can be selected in a sucrose-containing medium.

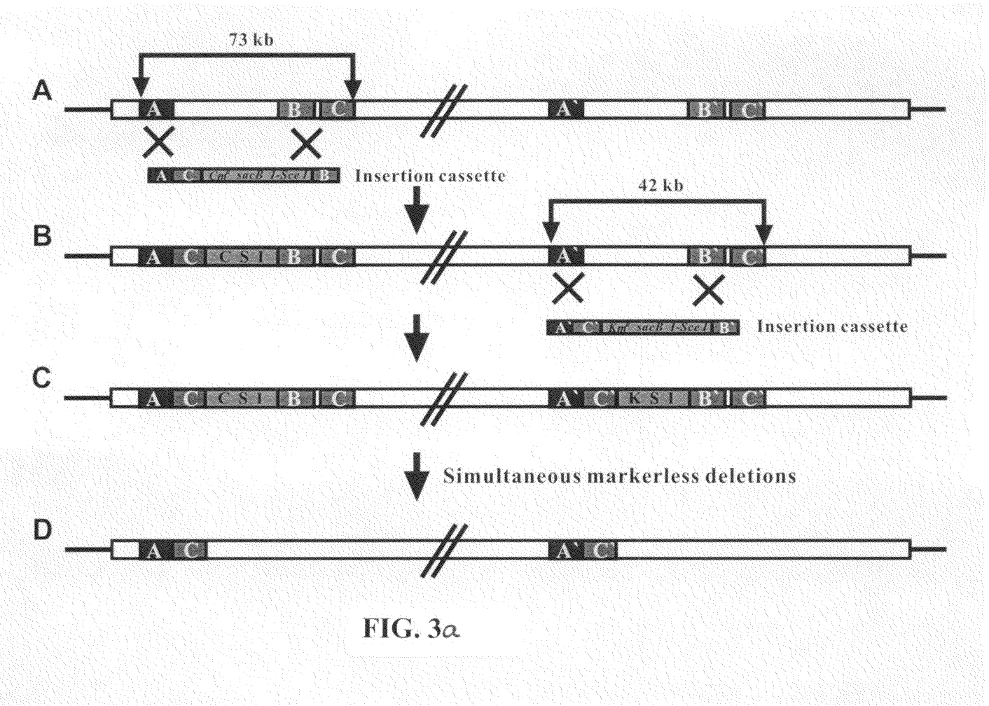

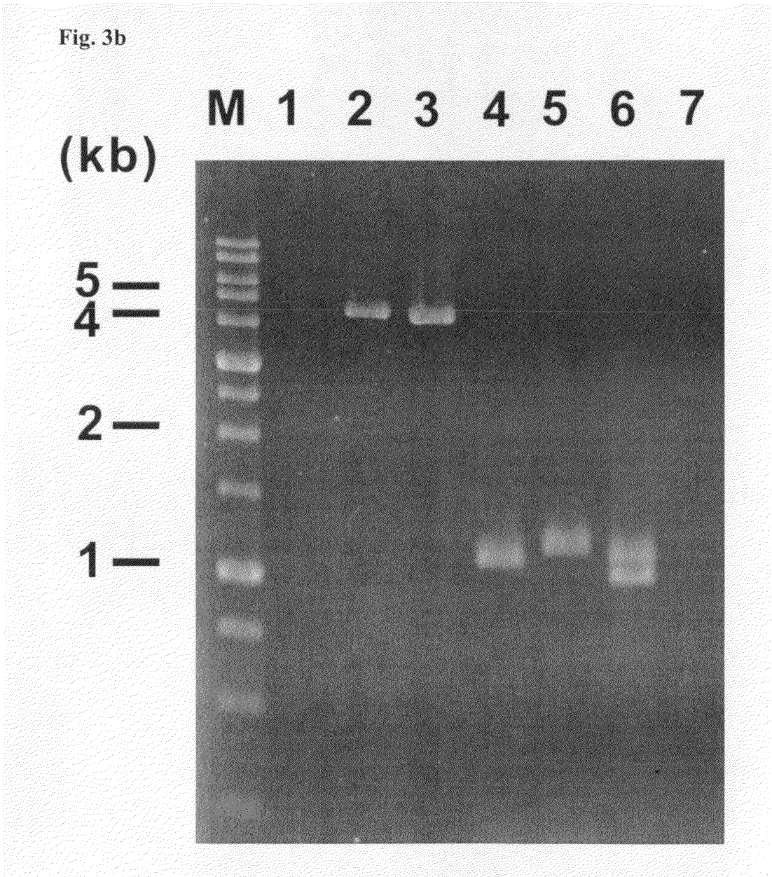

In order to delete two different genomic regions in Step 4 with modification of the above method, two linear DNA fragments containing different selectable markers, e.g. chloramphenicol-resistant gene (Cm^{R}) and kanamycin-resistant gene (Km^{R}), are constructed by PCR, as shown in FIG. 3a. A first linear DNA fragment is electroporated into the microbes transformed with the recombination vector of the present invention, a specific chromosomal locus of the microbial transformants is replaced with the linear DNA fragment through λ-red recombination of the recombination vector, the microbes are cultured and selected in a medium containing an antibiotic of interest corresponding to the kinds of selectable markers, and finally gene replacement on the chromosome of microbial strains is verified by PCR. Next, a second linear DNA fragment having another selectable marker is electroporated into the target strains, a specific chromosomal locus of the microbial transformants is replaced with the linear DNA through λ-red recombination, the microbes are cultured and selected in a medium containing a pertinent selectable marker, and finally gene replacement on the chromosome of the microbial strains is confirmed by PCR (FIG. 3b).

Further, it is possible to select microbial strains with deletion of two selectable markers as follows. First, the microbial strains having microbial chromosomal replacements with two linear DNA fragments are cultured in a rhamnose-containing medium to induce expression of an I-SceI gene which consequently results in specific cleavage of an I-SceI recognition site, thus leading to homologous recombination between the homology domains of the linear DNA fragment homologous to the microbial chromosome and the corresponding homologous microbial chromosomal region. Simultaneously with homologous recombination, a sacB gene of the linear DNA fragment introduced into the microbial mutant strains is expressed to induce cell toxicity. Therefore, the microbial mutants having homologous recombination-mediated deletion of two selectable markers can be selected in a sucrose-containing medium. In this manner, it is advantageous to greatly reduce the time necessary for deletion of targeted genomic regions through single-deletion procedure of the selectable markers recombinantly inserted into two different regions. Deletion of the selectable markers through homologous recombination can be verified by PCR (FIG. 3b). Even though the use of two linear DNA fragments was exemplified in the deletion of specific genomic regions, it is also possible to additionally delete multiple specific regions of the chromosome, using two or more linear DNA fragments.

As to Step 5 of the present invention method, deletion of a genomic region containing essential gene(s) is not feasible with a conventional genomic deletion strategy. In other words, the conventional method of deleting the essential gene-containing region disadvantageously involves division of the deletion target region into two genomic regions with respect to the essential gene and then deletion of the target gene by two deletion steps. Step (5) of the present invention method is intended to solve such a disadvantage. In order to achieve one-step deletion of a target genomic region having essential gene(s), a linear DNA fragment containing the essential gene(s) present in the target genomic region is constructed and the target genomic region is then replaced with the resulting linear DNA fragment construct using the recombination vector pREDI in accordance with the present invention.

As used herein, the term “essential gene” refers to a gene that is essentially necessary for survival of E. coli, which includes, for example, argS. Specifically, examples of 300 essential genes are disclosed in Baba T, Ara T, Hasegawa M, Takai Y, Okumura Y, Baba M, Datsenko K A, Torita M, Wanner B L, Mori H.; Construction of Escherichia coli K-12 in-frame, single-gene knockout mutants: the Keio collection.; Mol Syst Biol. 2006; 2:2006.0008. Epub2006 Feb. 21, page 5, line 9 (supplement data).

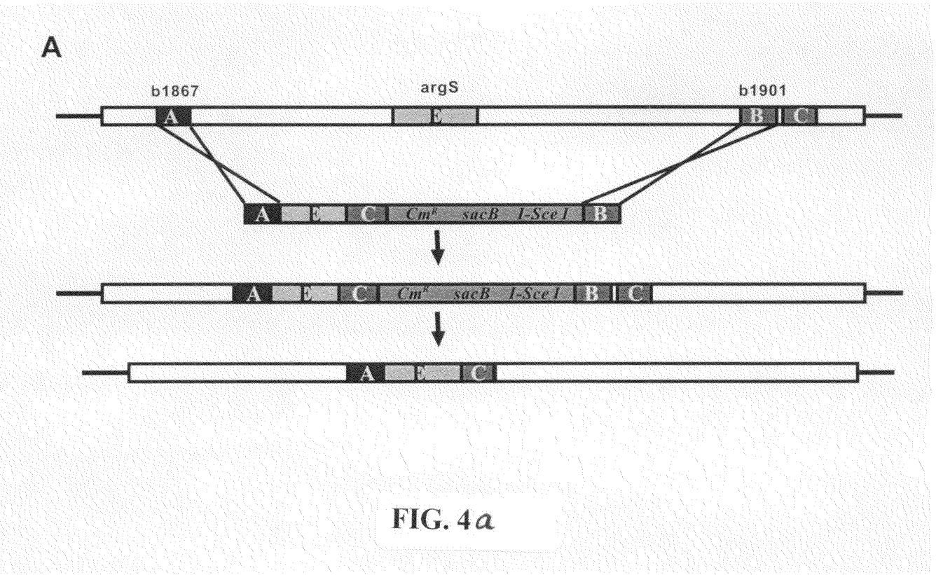

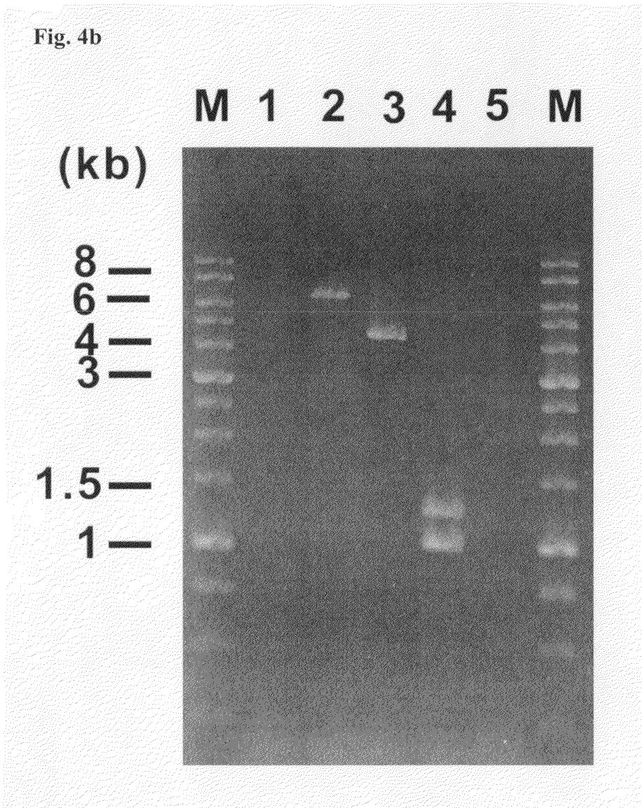

For example, as shown in FIG. 4a, a linear DNA fragment is constructed to contain an essential gene (E) (such as argS) between homology arms A and C and then a specific genomic region of E. coli is replaced with the resulting DNA fragment construct. As shown in Step 3, an E. coli strain with markerless deletion of a specific chromosomal region containing is selected by homologous recombination according to expression of I-SceI endonuclease and additionally, cell culture of E. coli in a sucrose-containing medium. In this manner, through the use of an essential gene-containing linear DNA fragment for deletion of a target genomic region having essential gene(s), it is possible to select E. coli strains having clean deletion of useless gene clusters from the selected genomic region while retaining the existing essential gene(s). Such genomic modifications can be verified by PCR (FIG. 4b).

As described before, it is possible to delete two or more specific gene regions or otherwise to successively delete specific gene regions of a microbial chromosome additionally containing essential gene(s), by repeating the steps of constructing a linear DNA fragment having a specific homology arm only and introducing the resulting construct into the target strain while rendering the strain to retain the recombination vector.

Even though E. coli is used as a subject microorganism, the present invention is not limited thereto. The linear DNA replacement is conducted based on the λ-red recombination method. The λ-red recombination-mediated replacement of an E. coli chromosome with a linear DNA fragment obtained by PCR amplification is carried out as described by Datsenko K A et al., PNAS, 97:6640. The homologous recombination process of deleting a selectable marker, which is promoted by cleavage of an E. coli chromosome, is conducted as described by Posfai G, et al., Nucleic Acids Res., 27:4409. 1999. In addition, use of the sacB gene as a selectable marker due to expression of toxicity to sucrose is disclosed in Van der Geize R, et al., FEMS Microbiol. Lett., 205:197, 2001.

Therefore, the present invention provides a rapid, efficient and markerless deletion method of a target genomic region which involves replacement of a specific locus of a microbial chromosome with a linear DNA fragment via λ-red recombination, subsequent removal of the selectable marker by I-SceI-mediated chromosomal cleavage in conjunction with induction of sacB-mediated sucrose toxicity, and finally expression of the gene under the control of two different promoters in the recombination vector pREDI. This method can be adapted for the construction of minimized genomes.

### EXAMPLES

Now, the present invention will be described in more detail with reference to the following Examples. These examples are provided only for illustrating the present invention and should not be construed as limiting the scope and spirit of the present invention.

### Example 1

### Construction of Recombinant Vector pREDI for Deletion of Specific Chromosomal Regions

A recombination vector in accordance with the present invention was constructed by cloning a recombinant PCR gene product rhaTS-Pᵣₕₐ-I-SceI fragment, which contains an NcoI recognition site and expresses I-SceI endonuclease under the control of rhamnose-inducible promoter (Pᵣₕₐ), into the NcoI site of a template vector pKD46 (Datsenko K A, et al., PNAS, 97:6640, 2000) which contains λ-red recombination functions under the control of an arabinose-inducible promoter (Pₐᵣₐ). Specifically, the rhaTS-Pᵣₕₐ-I-SceI fragment was constructed as follows. A PCR product rhaTS-Prha fragment was obtained by performing PCR amplification in an E. coli MG1655 genome using 25 pmoles of a primer NcoI-rha (SEQ ID NO: 9) and Pᵣₕₐ(SEQ ID NO: 10). A PCR product I-SceI fragment was obtained by performing PCR amplification in pST76-ASceP (Posfai G, et al., Nucleic Acids Res., 27:4409, 1999) using primers I-SceI-F (SEQ ID NO: 11) and NcoI-I-SceI (SEQ ID NO: 12). Then, the resulting rhaTS-Prha and I-SceI fragments were amplified by recombinant PCR, using primers NcoI-rha and NcoI-I-SceI. The resulting PCR product was digested with NcoI and cloned into the NcoI site of the plasmid pKD46 to thereby construct a recombination vector pREDI in accordance with the present invention. A cleavage map of the recombination vector pREDI is shown in FIG. 1. Base sequences of PCR primers are as follows:

Primer NcoI-rha (SEQ ID NO: 9):
5′-CATGCCATGGGGCATGGCGAATTAATCTTTCTG-3′
Primer Pᵣₕₐ (SEQ ID NO: 10):
5′-ATGTGATCCTGCTGAATTTCATTACGACCAGTCT-3′
Primer I-SceI-F (SEQ ID NO: 11):
5′-TTAGACTGGTCGTAATGAAATTCAGCAGGATCACATAATGCATCAAA
AAAAC CAGGTAATGAACCTGGG-3′
Primer NcoI-I-SceI (SEQ ID NO: 12):
5′-CATGCCATGGGTCGACTTATTATTTCAGGAAAGTTTCGGAGGAGATA
G-3,

### Example 2

### Deletion of Specific Chromosomal Regions of Microbes Using pREDI Vector (1) Construction of Linear DNA Fragments

From E. coli strain K-12 MG1655 (by courtesy of Dr. Jung-Hye Roe, Department of Microbiology, Seoul National University, Seoul, Korea), E. coli mutants where a useless gene cluster was deleted from the genome were obtained according to the following procedure.

First, the CM^{R} gene (SEQ ID NO: 13) of a pSG76 vector (Posfai G, et al., Nucleic Acid Res., 27:4409, 1999) was digested with two restriction endonucleases KpnI and BamHI (New England Biolabs, Beverly, Mass.) and cloned into the KpnI and BamHI sites of a pST76K vector (Posfai G, et al., Nucleic Acid Res., 27:4409, 1999) containing the I-SceI recognition site. Then, the sacB gene from a pDELTA vector (GibcoBRL-DELETION FACTORY SYSTEM VERSION 2.) was cleaved with BamHI (New England Biolabs, Beverly, Mass.) and ligated into the BamHI site of the above plasmid construct with the Cm^{R} gene and I-SceI recognition site, using a ligase (New England Biolabs, Beverly, Mass.). The constructed plasmid was designated as pSCI.

Further, the BamHI-cut sacB gene was ligated into the BamHI site of a pST76-K vector (Posfai G, et al., Nucleic Acid Res., 27:4409, 1999) containing the KM^{R} gene (SEQ ID NO: 14) and the I-SceI recognition site, using ligase (New England Biolabs, Beverly, Mass.). The resulting vector was designated as pSKI.

As shown in FIG. 2, linear DNA fragments (ca. 3.5 kb) containing three homology arms A (SEQ ID NO: 15), B (SEQ ID NO: 16) and C (SEQ ID NO: 17), and chloramphenicol-resistant gene (Cm^{R}, SEQ ID NO: 13), sacB (SEQ ID NO: 53) and I-SceI cleavage site (S, SEQ ID NO: 52) as selectable markers were constructed by recombinant PCR with an E. coli MG1655 chromosome and the above-constructed plasmid pSCI or pSKI as a template. One short (50 bp) terminal fragment (A: SEQ ID NO: 15) and one homology arm (500 bp) (C: SEQ ID NO: 17) were PCR-amplified using the E. coli K-12 MG1655 chromosome as a template and primers b0004A-F (SEQ ID NO: 18) and b0025C-R (SEQ ID NO: 19). The Cm^{R}-sacB-S fragment (CSI fragment) and the other short terminal fragment (B: SEQ ID NO: 16) were PCR-amplified using the plasmid pSCI as a template and primers CSI-f (SEQ ID NO: 20) and b0024B-R (SEQ ID NO: 21). Subsequently, the intact linear DNA fragments A-C-Cm^{R}-sacB-S-B were prepared by recombinant PCR, using A-C fragments and the PCR products Cm^{R}-sacB-S-B and Km^{R}-sacB-S-B as templates and primers b0004A-F (SEQ ID NO: 18) and b0024B-R (SEQ ID NO: 21). The thus-amplified DNAs were isolated using Nucleogen Gel-Extraction KIT (NucleoGen Inc., Gyeonggi-do, Korea). The linear DNA fragment obtained as above had a base sequence of SEQ ID NO: 54.

Base sequences of the PCR primers are as follows:

Primer b0004A-F (SEQ ID NO: 18):
5′-CCGCCGATTT TGCTGCGTTG CGTAAATTGA TGATGAATCA
TCAGTAAAAT CCA GAC ATG AAG CTG ATA CGC GGC-3′
Primer b0025C-R (SEQ ID NO: 19):
5′-CGA ATT CCG GTC TCC CTA TAG TGA GTC GTA CAG ATT
GTC ATC CGC AAG GGC CTG-3′
Primer CSI-F (SEQ ID NO: 20):
5′-TACGACTCACTATAGGGAGACCGGAATTCG-3′
Primer b0024B-R (SEQ ID NO: 21):
5′-CCC AGT ATA CCT TGT ACA GCG GTC AAG GTT AAC CGG
CGA TTG AGT ACC GA CTGCAGGTCGACTCTAGAGGATCT-3′

### (2) Deletion of Specific Genomic Regions of Microbes by Introduction of Linear DNA Fragments into E. coli Strain Transformed with pREDI Vector

The above-constructed linear DNA fragment was transferred into an E. coli strain MG1655 harboring the pREDI vector by a standard electroporation method (Bio-RAD, Bacterial electro-transformation and Plus Controller Instruction Manual, Cat. NO 165-2098; Thompson, J R, et al. Yeast 14:565, 1998; and Grant, S G, et al. Proc. Natl. Acas. Sci. USA, 87:4645, 1990). Since the linear DNA fragment contains two homology arms A and B that are homologous to the chromosomal region of the above E. coli strain, the genomic region (b0004-b0024) flanked by these two homology arms was deleted and then replaced with the linear DNA fragment. The E. coli mutants containing a gene replacement with the linear DNA fragment exhibit chloramphenicol-resistance due to the presence of a Cm^{R} gene in the linear DNA fragment, so they were selected in a chloramphenicol-containing medium.

Since the transcription of an I-SceI gene, which is present on the pREDI vector with selectable markers in the E. coli mutants having a gene cluster replacement of the E. coli b0004-b0024 region, is under the control of a rhamnose-inducible promoter, the E. coli mutants were cultured in a rhamnose-containing medium to thereby induce expression of the I-SceI endonuclease. By additional incorporation of sucrose into the culture medium during this process, bacterial selection was induced using a sacB gene present on the linear DNA fragment introduced into E. coli. That is, I-SceI-mediated restriction of the target microbial chromosomal region resulted in homologous recombination between the microorganism-derived region present on the introduced linear DNA fragment and the corresponding homologous region of the microbial chromosome, which leads to deletion of the selectable marker and sacB gene region from the E. coli chromosome, consequently rendering E. coli strains to lose levan toxicity. Therefore, the mutant strains with deletion of the selectable marker can be selected in a sucrose-containing medium (see FIG. 2). Deletion of the b0004-b0024 gene from the thus-selected mutant strains was confirmed by PCR. The mutant strain was designated as Δ(b0004-b0024).

### Example 3

### Deletion of Two Specific Genomic Regions Using Insertion and Deletion of Linear DNA Fragments Containing Two Different Selectable Markers into/from Specific Genomic Region

Two linear DNA fragments containing two different selectable markers were constructed. Analogously to Example 2, the constructed DNA fragments were sequentially replaced into the chromosome, followed by simultaneous deletion of two different genomic regions.

First, b0980-b1052 and b1137-b1168 regions of an E. coli genome were selected as two genomic regions to be deleted. For deletion of the b0980-b1052 region, a linear DNA fragment containing a chloramphenicol-resistant gene (Cm^{R}) was constructed analogously to Example 1, using homology arm A having a base sequence of SEQ ID NO: 22, homology arm B having a base sequence of SEQ ID NO: 23 and homology arm C having a base sequence of SEQ ID NO: 24, and primers b0980A-F (SEQ ID NO: 25), b0980A-R (SEQ ID NO: 26), b1051B-F (SEQ ID NO: 27), b1051B-R (SEQ ID NO: 28), b1052C-F (SEQ ID NO: 29) and b1052C-R (SEQ ID NO: 30). The resulting linear DNA fragment had a base sequence of SEQ ID NO: 55.

For deletion of the other region b1137-b1168, a linear DNA fragment containing a kanamycin-resistant gene (Km^{R}) was constructed using homology arm A having a base sequence of SEQ ID NO: 31, homology arm B having a base sequence of SEQ ID NO: 32 and homology arm C having a base sequence of SEQ ID NO: 33, and primers 1137A-F (SEQ ID NO: 34), 1137A-R (SEQ ID NO: 35), 1167B-F (SEQ ID NO: 36), 1167B-R (SEQ ID NO: 37), 1168C-F (SEQ ID NO: 38) and 1168C-R (SEQ ID NO: 39). The resulting linear DNA fragment had a base sequence of SEQ ID NO: 56. Base sequences of the primer as used herein are as follows.

Primer b0980A-F (SEQ ID NO: 25):
5′-ACTGTTGGTGTGATTCATCTGCG-3′
Primer b0980A-R (SEQ ID NO: 26):
5′-ACCCCAGGTATCGAACTGATTACCACATTGTTTCTGCTCCTTAG-3′
Primer b1051B-F (SEQ ID NO: 27):
5′-GGCTGATCAGCTAGCCCATGGGTATGAGCATTATTAGTCGCACTATA
CCG-3,
Primer b1051B-R (SEQ ID NO: 28):
5′-GATGACCATGTACGCAACGCTTG-3′
Primer b1052C-F (SEQ ID NO: 29):
5′-AATCAGTTCGATACCTGGGGT-3′
Primer b1052C-R (SEQ ID NO: 30):
5′-GCCGATCAACGTCTCATTTTCGCCAAAAATTCTGACGCTGTATGTCC
GCG-3,
Primer b1137A-F (SEQ ID NO: 34):
5′-CTGATGGATGGCGCTAAACTGCTG-3′
Primer b1137A-R (SEQ ID NO: 35):
5′-CGCAGGATCTCTTCAGGCGTTGAAGATGTATGTGAAGGGGCCGC-3′
Primer b1167B-F (SEQ ID NO: 36):
5′-GGCTGATCAGCTAGCCCATGGGTATGATCCAGCTCTGGTATTCCGC
A-3′
Primer b1167B-R (SEQ ID NO: 37):
5′-CCAATAATGTCATCCGCCACGCG-3′
Primer b1168C-F (SEQ ID NO: 38):
5′-CAACGCCTGAAGAGATCCTGCG-3′
Primer b1168C-R (SEQ ID NO: 39):
5′-GCCGATCAACGTCTCATTTTCGCCAAAAATTCGGGTTGATTCTGGGT
CTG-3′

As shown in FIG. 3a, in order to construct an E. coli mutant strain with deletions of the E. coli chromosomal regions b0980-b1052 and b1137-b1168, the b0980-b1052 region of E. coli was replaced with a linear DNA fragment (b0980-b1052+Cm^{R}) containing Cm^{R} as a selectable marker, analogously to Example 2, and then a second linear DNA fragment (b1137-b1168+Km^{R}) was also introduced to replace the b1137-b1168 region of the E. coli chromosome, analogously to Example 2. The E. coli mutants having chromosomal replacements with two linear DNA fragments exhibit chloramphenicol and kanamycin resistance due to the presence of Cm^{R} and Km^{R} genes on the linear DNA fragments, so these E. coli strains were selected in a medium supplemented with chloramphenicol and kanamycin.

Thereafter, the selectable marker genes Cm^{R} and Km^{R} remaining in the E. coli mutant strains with deletions of b0980-b1052 and b1137-b1168 were simultaneously deleted analogously to Example 2, thereby constructing a mutant strain from which the E. coli b0980-b1052 and b1137-b1168 were deleted. Deletion of the selected genomic regions was confirmed by PCR. In FIG. 3b, M represents a marker, Lanes 1 to 3 are intended to confirm the incorporation of linear DNA fragments into E. coli, wherein Lane 1 represents a non-introduced MG1655 strain (positive control), Lane 2 represents PCR results to confirm introduction of a DNA fragment A-C-Cm sacB I-SecI-B constructed to delete the b0980-b1052 region, Lane 3 represents PCR results to confirm introduction of a DNA fragment A-C-Km sacB I-SecI-B constructed to delete the b1137-b1168 region, Lane 4 represents PCR results to confirm I-SecI-mediated deletion of a Cm sacB I-SecI-B region after introduction of a DNA fragment A-C-Cm sacB I-SecI-B constructed to delete the b0980-b1052 region, Lane 5 represents PCR results to confirm I-SecI-mediated deletion of a Km sacB I-SecI-B region after introduction of a DNA fragment A-C-Km sacB I-SecI-B constructed to delete the b1137-b1168 region, and Lanes 6 and 7 represent PCR results for each region between b0980-b1052 and between b1137-b1168, conducted to confirm whether the b0980-b1052 and b1137-b1168 regions were deleted from the E. coli strain. Lane 6 is an E. coli strain with non-deletion of the b0980-b1052 and b1137-b1168 regions, e.g. MG1655 strain having no deletion of target genomic regions similar to the sample of Lane 1, thereby showing a band. On the other hand, Lane 7 exhibits no characteristic band due to deletion of b0980-b1052 and b1137-b1168. The efficiency of simultaneous deletion of specific genomic regions was nearly 95%.

FIG. 3a shows a markerless deletion process of inserting two linear DNA fragments containing different selectable markers into two specific genomic regions of E. coli and simultaneously deleting the specific genomic regions.

### Example 4

### Deletion of Specific Chromosomal Regions Containing Essential Gene(s) from Microbes

A useless genomic cluster containing essential gene(s) necessary for survival of microbes cannot be deleted with a conventional genomic deletion technique. The conventional gene deletion method of the essential gene-containing region was disadvantageously conducted including division of a deletion-targeted specific region into two genomic regions with respect to the essential gene and two deletion operations of the target region. In order to solve such a disadvantage of the conventional method, only the desired chromosomal region was deleted while retaining the essential gene, using the recombination vector constructed in Examples of the present invention.

For this purpose, a specific genomic region yecD-araF (b1867-b1901) of E. coli containing an essential gene argS (b1876) encoding an arginyl-tRNA synthetase was selected. For deletion of the specific genomic region yecD-araF (b1867-b1901), a linear DNA fragment having a structure of [homology arm A (SEQ ID NO: 40)-E(argS)-homology arm C (SEQ ID NO:42)-Cm^{R}-sacB-I-SceI-homology arm B (SEQ ID NO: 41)] as shown in FIG. 4a was constructed according to the method disclosed in Example 1. The resulting linear DNA fragment had a base sequence of SEQ ID NO: 57.

In order to replace the b1867-b1901 genomic region, the above-constructed DNA fragment was introduced into the E. coli strains. Then, the E. coli strains with a deletion of yecD-araF (b1867-b1901) containing argS (b1876) were cultured and selected in a chloramphenicol-containing medium. According to the method disclosed in Example 2, desired strains, exhibiting traceless deletion of the specific chromosomal region through homologous recombination while removing the selectable marker that was contained in the linear DNA fragment, were successfully selected. FIG. 4a shows a process illustrating deletion of a specific genomic region containing an essential gene. Replacement and removal of the linear DNA fragment into/from the E. coli chromosome were confirmed by PCR, using primers specific for a gene of the specific region to be deleted. In FIG. 4b, M: Marker, Lane 1: MG1655 strain not transformed with the linear DNA fragment, Lane 2: MG1655 strain transformed with the linear DNA fragment, thus confirming whether the targeted genomic region of the strain was replaced with the linear DNA fragment, Lane 3: PCR results confirming I-SceI-mediated deletion of a Cm^{R}-sacB-I-SceI-homology arm B portion from a DNA fragment consisting of homology arm A-E(argS)-homology arm C-Cm^{R}-sacB-I-SceI-homology arm B, Lane 4: MG1655 strain with non-deletion of the specific chromosomal region, which is a sample similar to that of Lane 1. Appearance of two bands is due to simultaneous PCR of two regions between b1867-b1901. Lane 5: MG1655 strain with deletion of the specific chromosomal region and exhibiting no characteristic band, thus representing that the specific chromosomal region was correctly deleted. Instead of carrying out two deletion steps, i.e. one operation at each side with respect to the essential gene, the aforesaid method of the present invention enables advantageously rapid and efficient deletion of the targeted specific region even with single-deletion operation. Base sequences of primers as used herein are as follows.

Primer b1867A-F (SEQ ID NO: 44):
5′-TGCGCTGGTGGTGATCGATTTAC-3′
Primer b1867A-R (SEQ ID NO: 45):
5′-TGGCACAGGGCAACAGGGTAAACGCGTTGAGGATCTCTTCCACG-3′
Primer argS-F (SEQ ID NO: 46):
5′-TTACCCTGTTGCCCTGTGCCA-3′
Primer argS-R (SEQ ID NO: 47):
5′-GCGATGTGATATTGCTCTCCTATGGAGATCGGCTAACCCTGATCA
GGCTTC-3′
Primer b1901C-F (SEQ ID NO: 48):
5′-CTCCATAGGAGAGCAATATCACATCGC-3′
Primer b1901C-R (SEQ ID NO: 49):
5-GCCGATCAACGTCTCATTTTCGCCAAAAATGTGATCGTGGAGTCAA
TTCTGACG-3′
Primer b1900B-F (SEQ ID NO: 50):
5′-GGCTGATCAGCTAGCCCATGGGTATGACCAGCATTGAGTTGGCAGC
G-3′
Primer b1900B-R (SEQ ID NO: 51):
5′-ATGTCACAATCCGCTATGGCGG-3′

As apparent from the above description, the recombinant vector for deletion of specific chromosomal regions in accordance with the present invention is capable of conveniently and rapidly achieving successive deletion of targeted specific genes with only one vector, as compared to conventional gene deletion methods involving the use of multiple vectors. In addition, the present invention enables prompt and efficient deletion of target genomic regions containing essential gene(s) in a single-step fashion.

What is claimed is:

1. A recombinant vector for deletion of specific chromosomal regions, comprising an arabinose-inducible promoter (Pₐᵣₐ); a gene encoding a protein involved in lambda (λ)-red recombination; a rhamnose-inducible promoter (Pᵣₕₐ); and a gene encoding the I-SceI endonuclease, wherein the vector has a base sequence of SEQ ID NO: 1 and is represented by a cleavage map of FIG. 1.

2. Escherichia coli transformed with the recombination vector of claim 1.

3. A method for deletion of specific chromosomal regions of a microbe using the recombination vector of claim 1, comprising the steps of:
1) preparing a linear DNA fragment containing homology arms A and B which are involved in λ-red recombination when they are introduced into a target microbe; a selectable marker; an I-SceI recognition site which is involved in homologous recombination for removal of the selectable marker; and a homology arm C which is involved in homologous recombination for removal of the selectable marker;
2) introducing the linear DNA fragment into a microbe transformed with the recombination vector of claim 1 to replace a specific locus of the microbial chromosome with the linear DNA fragment through λ-red recombination between the homology arms of the DNA fragment and the microbial chromosome regions homologous to the homology arms; and
3) culturing the specific chromosomal locus-replaced microbe in a rhamnose-containing medium to induce expression of the I-SceI endonuclease, such that homologous recombination between the homology arm C of the DNA fragment and the microbial chromosomal region homologous to the homology arm C is driven to remove the selectable marker,
wherein the homology arm A is a region homologous to 50 to 500 bp of one end of the deletion target domain of a microbial chromosome and the homology arm B is a region homologous to 50 to 500 bp of the other end of the deletion target domain of the microbial chromosome, and
the homology arm C is a region that is homologous to a 300-500 bp region contiguous to either one of the microbial chromosome regions homologous to the homology arm A and homology arm B.

4. The method according to claim 3, wherein the method includes repeating Steps 1 and 2 to prepare a plurality of different linear DNA fragments and introducing the linear DNA fragments into microbes transformed with a recombination vector of claim 1 to delete a plurality of specific microbial chromosomal regions.

5. The method according to claim 3, wherein the selectable marker is at least one selected from the group consisting of a chloramphenicol-resistant gene having a base sequence of SEQ ID NO: 13, a kanamycin-resistant gene having a base sequence of SEQ ID NO: 14, and sacB.

6. The method according to claim 5, further comprising culturing the microbes in a sucrose-containing medium after the step of removing the selectable marker.

7. The method according to claim 3, wherein the homology arm A, the homology arm B and the homology arm C have base sequences of SEQ ID NO: 15, SEQ ID NO: 16 and SEQ ID NO: 17, respectively.

8. The method according to claim 3, wherein the homology arm A, the homology arm B and the homology arm C have base sequences of SEQ ID NO: 22, SEQ ID NO: 23 and SEQ ID NO: 24, respectively.

9. The method according to claim 3, wherein the homology arm A, the homology arm B and homology arm C have base sequences of SEQ ID NO: 31, SEQ ID NO: 32 and SEQ ID NO: 33, respectively.

10. The method according to claim 3, wherein the specific chromosomal region of the microbe contains a gene essential for survival of the microbe, and the linear DNA fragment further contains the essential survival gene between the homology arm A and the homology arm C.

11. The method according to claim 10, wherein the essential gene is argS having a base sequence of SEQ ID NO: 43, and the homology arm A, the homology arm B and the homology arm C have base sequences of SEQ ID NO: 40, SEQ ID NO: 41 and SEQ ID NO: 42, respectively.


SEQUENCE LISTING (57 sequences)

SEQ ID NO: 1 - DNA, 9036 bp, Artificial Sequence
  pREDI vector
catcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtcgacttattatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggttagccagtttgttgaaagcttggtgtttgaaagacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggtttttttgatgcatatgtgatcctgctgaatttcattacgaccagtctaaaaagcgcctgaattcgcgaccttctcgttactgacaggaaaatgggccattggcaaccagggaaagatgaacgtgatgatgttcacaatttgctgaattgtggtgatgtgatgctcaccgcatttcctgaaaattcacgctgtatcttgaaaaatcgacgttttttacgtggttttccgtcgaaaatttaaggtaagaacctgacctcgtgattactatttcgccgtgttgacgacatcaggaggccagtatgaccgtattacatagtgtggatttttttccgtctggtaacgcgtccgtggcgatagaaccccggctcccgcaggcggattttcctgaacatcatcatgattttcatgaaattgtgattgtcgaacatggcacgggtattcatgtgtttaatgggcagccctataccatcaccggtggcacggtctgtttcgtacgcgatcatgatcggcatctgtatgaacataccgataatctgtgtctgaccaatgtgctgtatcgctcgccggatcgatttcagtttctcgccgggctgaatcagttgctgccacaagagctggatgggcagtatccgtctcactggcgcgttaaccacagcgtattgcagcaggtgcgacagctggttgcacagatggaacagcaggaaggggaaaatgatttaccctcgaccgccagtcgcgagatcttgtttatgcaattactgctcttgctgcgtaaaagcagtttgcaggagaacctggaaaacagcgcatcacgtctcaacttgcttctggcctggctggaggaccattttgccgatgaggtgaattgggatgccgtggcggatcaattttctctttcactgcgtacgctacatcggcagcttaagcagcaaacgggactgacgcctcagcgatacctgaaccgcctgcgactgatgaaagcccgacatctgctacgccacagcgaggccagcgttactgacatcgcctatcgctgtggattcagcgacagtaaccacttttcgacgctttttcgccgagagtttaactggtcaccgcgtgatattcgccagggacgggatggctttctgcaataacgcgaatcttctcaacgtatttgtacgccatattgcgaataatcaacttcgttctctggccgaggtagccacggtggcgcatcagttaaaacttctcaaagatgatttttttgccagcgaccagcaggcagtcgctgtggctgaccgttatccgcaagatgtctttgctgaacatacacatgatttttgtgagctggtgattgtctggcgcggtaatggcctgcatgtactcaacgatcgcccttatcgcattacccgtggcgatctcttttacattcatgctgacgataaacactcctacgcttccgttaacgatctggttttgcagaatattatttattgcccggagcgtctgaagctgaatcttgactggcagggggcgattccgggatttaacgccagcgcagggcaaccacactggcgcttaggtagcatggggatggcgcaggcgcggcaggttatcggtcagcttgagcatgaaagtagtcagcatgtgccgtttgctaacgaaatggctgagttgctgttcgggcagttggtgatgttgctgaatcgccatcgttacaccagtgattcgttgccgccaacatccagcgaaacgttgctggataagctgattacccggctggcggctagcctgaaaagtccctttgcgctggataaattttgtgatgaggcatcgtgcagtgagcgcgttttgcgtcagcaatttcgccagcagactggaatgaccatcaatcaatatctgcgacaggtcagagtgtgtcatgcgcaatatcttctccagcatagccgcctgttaatcagtgatatttcgaccgaatgtggctttgaagatagtaactatttttcggtggtgtttacccgggaaaccgggatgacgcccagccagtggcgtcatctcaattcgcagaaagattaattcgccatgccccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 2 - DNA, 1233 bp, Artificial Sequence
  arabinose promoter
ttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaa

SEQ ID NO: 3 - DNA, 1885 bp, Artificial Sequence
  gamma-RED recombination System_gamma,beta,exo gene
atggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatga

SEQ ID NO: 4 - DNA, 417 bp, Artificial Sequence
  lambda-RED recombination System gamma gene
atggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataa

SEQ ID NO: 5 - DNA, 786 bp, Artificial Sequence
  lambda-RED recombination System beta gene
atgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatga

SEQ ID NO: 6 - DNA, 681 bp, Artificial Sequence
  lambda-RED recombination System exo gene
atgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatga

SEQ ID NO: 7 - DNA, 2056 bp, Artificial Sequence
  rhamnose promoter
ggcatggcgaattaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaaattcagcaggatcacat

SEQ ID NO: 8 - DNA, 639 bp, Artificial Sequence
  I-SceI restriction enzyme
atgcatcaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtctttcaaacaccaagctttcaacaaactggctaacctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataataa

SEQ ID NO: 9 - DNA, 33 bp, Artificial Sequence
  primer NcoI-rha
catgccatggggcatggcgaattaatctttctg

SEQ ID NO: 10 - DNA, 34 bp, Artificial Sequence
  primer prha
atgtgatcctgctgaatttcattacgaccagtct

SEQ ID NO: 11 - DNA, 69 bp, Artificial Sequence
  primer I-SceI-F
ttagactggtcgtaatgaaattcagcaggatcacataatgcatcaaaaaaaccaggtaatgaacctggg

SEQ ID NO: 12 - DNA, 48 bp, Artificial Sequence
  primer NcoI-I-SceI
catgccatgggtcgacttattatttcaggaaagtttcggaggagatag

SEQ ID NO: 13 - DNA, 660 bp, Artificial Sequence
  Chloramphenicol resistant gene
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccgaaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 14 - DNA, 795 bp, Artificial Sequence
  Kanamycin resistant gene
tcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatcgggcatc

SEQ ID NO: 15 - DNA, 50 bp, Artificial Sequence
  homology arm A for b0004-b0024 deletion
ccgccgattttgctgcgttgcgtaaattgatgatgaatcatcagtaaaat

SEQ ID NO: 16 - DNA, 50 bp, Artificial Sequence
  homology arm B for b0004-b0024 deletion
ccgagtataccttgtacagcggtcaaggttaaccggcgattgagtaccga

SEQ ID NO: 17 - DNA, 540 bp, Artificial Sequence
  homology arm C for b0004-b0024 deletion
ccagacatgaagctgatacgcggcatacataatctcagccaggccccgcaagaagggtgtgtgctgactattggtaatttcgacggcgtgcatcgcggtcatcgcgcgctgttacagggcttgcaggaagaagggcgcaagcgcaacttaccggtgatggtgatgctttttgaacctcaaccactggaactgtttgctaccgataaagccccggcaagactgacccggctgcgggaaaaactgcgttaccttgcagagtgtggcgttgattacgtgctgtgcgtgcgtttcgacaggcgtttcgcggcgttaaccgcgcaaaatttcatcagcgatcttctggtgaagcatttgcgcgtaaaatttcttgccgtaggtgatgatttccgctttggcgctggtcgtgaaggcgatttcttgttattacagaaagctggcatggaatacggcttcgatatcaccagtacgcaaactttttgcgaaggtggcgtgcgcatcagcagcaccgccgtgcgtcaggcccttgcggatgacaatctg

SEQ ID NO: 18 - DNA, 74 bp, Artificial Sequence
  primer b0004A-F
ccgccgattttgctgcgttgcgtaaattgatgatgaatcatcagtaaaatccagacatgaagctgatacgcggc

SEQ ID NO: 19 - DNA, 54 bp, Artificial Sequence
  primer b0025C-R
cgaattccggtctccctatagtgagtcgtacagattgtcatccgcaagggcctg

SEQ ID NO: 20 - DNA, 30 bp, Artificial Sequence
  primer CSI-f
tacgactcactatagggagaccggaattcg

SEQ ID NO: 21 - DNA, 74 bp, Artificial Sequence
  primer b0024B-R
ccgagtataccttgtacagcggtcaaggttaaccggcgattgagtaccgactgcaggtcgactctagaggatct

SEQ ID NO: 22 - DNA, 583 bp, Artificial Sequence
  homology arm A for b0980-b1052 deletion
actgttggtgtgattcatctgcgttcacagctggcgaccaaacgcgctgcactgttggtgatgctgtgctttttgctggcgggttactggctgtgggtcggtattgatggctttgtactgctcgcccaggatgctaacggtccttccaatccgttaatgaaactggtggcagtgctacctggtgcctggatgaataattttgtcgagtcgcccgttttgtggatcttcccgctgctgggattcttctgcccattgctgacggtgatggcgatttatcgtggtcgcccgggttggggatttttgatggcatcattgatgcaatttggcgtgattttcacggcaggcatcacgctgttcccctttgtcatgccgtcaagcgtgagtccgatctccagcctgacgttgtgggacagtacttccagtcagctgacgctgagcattatgttggtaatcgtgctgatatttttgcccattgtgttgctctacactctctggagctactacaaaatgtgggggcgcatgacaacagaaactctccgccgtaacgaaaacgagttgtactaaggagcagaaacaatgtggt

SEQ ID NO: 23 - DNA, 553 bp, Artificial Sequence
  homology arm B for b0980-b1052 deletion
agcattattagtcgcactataccggggcaggggcatggcaaccaatattatcccggtgtgcaatgggatgtgcgtgactccgcctggcgttatgtcacgatccttgatctgccattctctctggtttttgatactttactgctgccgatcgacattcatcatggcccgtatgagtgattaacgttcatcccactcatcagctgctgcgcgtccttcctcggtatccagcggtggctcaagctgaaattccccctcgtcccattcatgtaatgtattctcttcctgccactcctggcgtatctctatctcatcatagtcgccatcaaaaacactttgcgcggcttcaccgctaagcataggtaaacattcaccttcttccccttcgtcggcaaaaaactcaacttgccacatgatgtcgccgtcctgcaaaacgtatttttgggcattgaactgttgcacattcgcatcttcggcgtcgatgccggggttgtctgcaagaaattcttcgcgtgcagcgtcaatggcttcttcaagcgttgcgtacatggtcatc

SEQ ID NO: 24 - DNA, 509 bp, Artificial Sequence
  homology arm C for b0980-b1052 deletion
aatcagttcgatacctggggtattcgacgacgacgtagactgttccatgaatagactgcgttgaataacactacgccagcggtgacgagaaataccgctctgaaaccgtagttcgctgaaatcgctgctcccatcaatggtccggtaacgttgccaatatcacgaaacgattggttatagctgaagatacgcccggcgatctggttgctcgagttgtaaaccaacagtgtctgtacggcggggagtagtgcaccatcggcggcaccgagcaaaaaacgtaaaatcccaagttgcaatggcgtctgaacgtaagacattgggatcaacagcagtacagaaaagatcagcgctgtaatcaggatcttttcgggtccgattcgatcgccaagtttgccgagtcgtggtgcacttagcagagccgccacgcctggcaccgaggcgatcatgccactgataaaggcgacgttactgacgttacccgccagttcgcggacatacagcgtcagaat

SEQ ID NO: 25 - DNA, 23 bp, Artificial Sequence
  primer b0980A-F
actgttggtgtgattcatctgcg

SEQ ID NO: 26 - DNA, 44 bp, Artificial Sequence
  primer b0980A-R
accccaggtatcgaactgattaccacattgtttctgctccttag

SEQ ID NO: 27 - DNA, 50 bp, Artificial Sequence
  primer b1051B-F
ggctgatcagctagcccatgggtatgagcattattagtcgcactataccg

SEQ ID NO: 28 - DNA, 23 bp, Artificial Sequence
  primer b1051B-R
gatgaccatgtacgcaacgcttg

SEQ ID NO: 29 - DNA, 21 bp, Artificial Sequence
  primer b1052C-F
aatcagttcgatacctggggt

SEQ ID NO: 30 - DNA, 50 bp, Artificial Sequence
  primer b1052C-R
gccgatcaacgtctcattttcgccaaaaattctgacgctgtatgtccgcg

SEQ ID NO: 31 - DNA, 520 bp, Artificial Sequence
  homology arm A for b1137-b1158 deletion
ctgatggatggcgctaaactgctgaaatgttcagagtttggtgacgcgatcatcgaaaacatgtaatgccgtagtttgttaaatttattaacgggagcgtaacgctcccgttgttttttgttaggctgctaacggttatcaaaattttatcaaaaaaagttatcaaaacccctcggtagttttggggtaggctggccggtcaggtggtagttctactactagtctcccacatagatattccttagctttttattattgctggcggacgctcgttaatatttaaggtcttcattgattaagacatccccaaagttagttatgtattcactgttattaggacaattatgaattaccactccttacacccgctcaaatattgttaaattgccggttttgtatcaactactcacccgggactcgccaggggacagccaacaggcattgggtgcaatcaccttagcgttcaggtacatgcggaatgtaaaaaaggccgcgagcgcggccccttcacatacatctt

SEQ ID NO: 32 - DNA, 461 bp, Artificial Sequence
  homology arm B for b1137-b1158 deletion
atccagctctggtattccgcaaaagcagagtacctggcgggagcgagatatgccgccaacaatatcaatcatatacttgaagaagcgtcacaagcgactcaaacagcggttaacattgccgggaaggaatgcaacctcgaggagcaatatcagcttggcactgaagcagctctgaaacctcacctgcgcacaatcatcattctcaaacagggaatagtctggtgtacatccctgcctgggaatcgggtcctgttgtctcgtattcctgttttcccggacagtaatttactgttggctccagcaatcgacaccgttaatagattacctatcctgctctatcagaaccaatttgcagatacgcgcattttggttacgataagcgatcagcatattcgtggggcacttaatgtacccttgaaaggggtaaggtatgtattacgcgtggcggatgacattattgg

SEQ ID NO: 33 - DNA, 583 bp, Artificial Sequence
  homology arm C for b1137-b1158 deletion
caacgcctgaagagatcctgcgacgggcaataaataatggggagatcgtccctttttaccaacctgtggtaaatggtcgggaagggacattgcggggagttgaggtgttagcccgctggaaacaacctcacggtggatatatatcacccgcggcatttattccacttgctgaaaaatccggattaatcgttccgcttacgcaaagcctgatgaatcaggttgccagacagatgaacgctatcgcgagtaaattgccggaaggttttcatattggaattaattttagcgcttcgcatattatttcgccgacgtttgtcgacgagtgtttaaatttccgtgacagttttacccgccgcgatttaaaccttgttctggaagtcaccgagcgtgagccattgaatgttgatgaaagtctggttcagcggttgaacatactgcatgaaaatggttttgtcatcgcactggatgatttcggtactggctactcagggctttcttatcttcatgacctgcatattgattatatcaaaattgatcacagtttcgttggccgcgtaaacgcagacccagaatcaacccgaat

SEQ ID NO: 34 - DNA, 24 bp, Artificial Sequence
  primer b1137A-F
ctgatggatggcgctaaactgctg

SEQ ID NO: 35 - DNA, 44 bp, Artificial Sequence
  primer b1137A-R
cgcaggatctcttcaggcgttgaagatgtatgtgaaggggccgc

SEQ ID NO: 36 - DNA, 47 bp, Artificial Sequence
  primer b1167B-F
ggctgatcagctagcccatgggtatgatccagctctggtattccgca

SEQ ID NO: 37 - DNA, 23 bp, Artificial Sequence
  primer b1167B-R
ccaataatgtcatccgccacgcg

SEQ ID NO: 38 - DNA, 22 bp, Artificial Sequence
  primer b1168C-F
caacgcctgaagagatcctgcg

SEQ ID NO: 39 - DNA, 50 bp, Artificial Sequence
  primer b1168C-R
gccgatcaacgtctcattttcgccaaaaattcgggttgattctgggtctg

SEQ ID NO: 40 - DNA, 536 bp, Artificial Sequence
  homology arm A for b1867-b1901 deletion
tgcgctggtggtgatcgatttacaagaaggcatcttaccttttgccggtggtccacatactgccgatgaggtagttaatcgcgccgggaagctggcggcgaaatttcgcgccagcggtcagcccgtgtttctggtgcgcgttggctggtctgccgattacgctgaagcattaaaacagccggtcgatgcgccctcaccggctaaagtgttgcccgaaaattggtggcaacatcctgctgcattaggtgcaaccgacagcgatatcgaaatcatcaaacgtcaatggggtgcgttttacggtacggatctggagttgcaattacgccgccgaggtatcgatacaatagtgttatgtgggatctcgaccaatatcggtgttgaatccaccgcccgcaatgcctgggaacttggctttaatctggtgattgccgaagacgcctgtagtgccgccagcgccgagcagcacaataacagtatcaaccatatctacccgcgcatcgcccgtgtgcgtagcgtggaagagatcctcaacgcgt

SEQ ID NO: 41 - DNA, 593 bp, Artificial Sequence
  homology arm B for b1867-b1901 deletion
accagcattgagttggcagcgtcaaatgcccccgggatgtcgttagatttggtaggtacctgataaatttgtttttccgggaatccggccgctttcagcgcatccatagatcccgtagtacggcggcgggcggtatccagttcgttggcggtaatcgccatcaccgcgctttctttgacatcccagccacgtttctgcatctctttatacagttcctggccctgacgttcgccaattttagtcgccgccatcatcaccagcggaacggtatccattggcttacctttggcgttaacaaactggtcatccacggcaatgactttcatatcgtagccacgcgctttcgcgacgatggcagagccgagtttggggtccggagtacaaataacgaaaccttttgcgccactggcagccaggctgtcgatcgcgttcaatgttttttcgccatccggcacggcaatcttaataacctcaaaccctaaatccttcccggctttatcggcaaacttccattcggtctggaaccacggctcttccggttgcttcaccagaaaaccgagcttcaggttctccgccatagcggattgtgacat

SEQ ID NO: 42 - DNA, 564 bp, Artificial Sequence
  homology arm C for b1867-b1901 deletion
ctccataggagagcaatatcacatcgcagaattacagtgagaacgtgcataaatttagcgggaaaagacataagggaaagccaatttgtcagacaaattgtcgaatgcacagcagattaatccataagattagcctggaaatccttgttgtctttggtacccatgcgggatgtcttctttttaaccagtcaataggccgcattacctggcgttgagtttttgaaatggtgtaataaccgcaactcaaagatgtggaaaatgcacgtcattcatttcgtcattaattatcactgtgctcattaattaacagaacacgtataatgagagccatctcgcaaaaatgaaaaaacgttttataaaatcatcacttcatcatgaattcaaattcattgattaatatcaacaagatacaaaaagcactatcattaaaattcattgcagttacattgatttcatcaatgaaatgtaaaaatatataaacttgatgatttaagcattttcttatacccgttcagacgttattcttatttcagatcatcgtcagaattgactccacgatcacat

SEQ ID NO: 43 - DNA, 1958 bp, Artificial Sequence
  argS gene
ttaccctgttgccctgtgccaaccaaccgctgatttcacgccgcttctgatgcaatagtgaaaacggcaatacgccacgcgcacgttgctgacgaaaacagccatttgcagtatactcccgccctaatttctttaactggtgcgggcaatttttgctcgcttcatcaatgtaaggtattccggtgaatattcaggctcttctctcagaaaaagtccgtcaggccatgattgcggcaggcgcgcctgcggattgcgaaccgcaggttcgtcagtcagcaaaagttcagttcggcgactatcaggctaacggcatgatggcagttgctaaaaaactgggtatggcaccgcgacaattagcagagcaggtgctgactcatctggatcttaacggtatcgccagcaaagttgagatcgccggtccaggctttatcaacattttccttgatccggcattcctggctgaacatgttcagcaggcgctggcgtccgatcgtctcggtgttgctacgccagaaaaacagaccattgtggttgactactctgcgccaaacgtggcgaaagagatgcatgtcggtcacctgcgctctaccattattggtgacgcagcagtgcgtactctggagttcctcggtcacaaagtgattcgcgcaaaccacgtcggcgactggggcactcagttcggtatgctgattgcatggctggaaaagcagcagcaggaaaacgccggtgaaatggagctggctgaccttgaaggtttctaccgcgatgcgaaaaagcattacgatgaagatgaagagttcgccgagcgcgcacgtaactacgtggtaaaactgcaaagcggtgacgaatatttccgcgagatgtggcgcaaactggtcgacatcaccatgacgcagaaccagatcacctacgatcgtctcaacgtgacgctgacccgtgatgacgtgatgggcgaaagcctctacaacccgatgctgccaggaattgtggcggatctcaaagccaaaggtctggcagtagaaagcgaaggggcgaccgtcgtattccttgatgagtttaaaaacaaggaaggcgaaccgatgggcgtgatcattcagaagaaagatggcggctatctctacaccaccactgatatcgcctgtgcgaaatatcgttatgaaacactgcatgccgatcgcgtgctgtattacatcgactcccgtcagcatcaacacctgatgcaggcatgggcgatcgtccgtaaagcaggctatgtaccggaatccgtaccgctggaacaccacatgttcggcatgatgctgggtaaagacggcaaaccgttcaaaacccgcgcgggtggtacagtgaaactggccgatctgctggatgaagccctggaacgtgcacgccgtctggtggcagaaaagaacccggatatgccagccgacgagctggaaaaactggctaacgcggttggtattggtgcggtgaaatatgcggatctctccaaaaaccgcaccacggactacatcttcgactgggacaacatgctggcgtttgagggtaataccgcgccatacatgcagtatgcatacacgcgtgtattgtccgtgttccgtaaagcagaaattgacgaagagcaactggctgcagctccggttatcatccgtgaagatcgtgaagcgcaactggcagctcgcctgctgcagtttgaagaaaccctcaccgtggttgcccgtgaaggcacgccgcatgtaatgtgtgcttacctgtacgatctggccggtctgttctctggcttctacgagcactgcccgatcctcagcgcagaaaacgaagaagtgcgtaacagccgtctaaaactggcacaactgacggcgaagacgctgaagctgggtctggatacgctgggtattgagactgtagagcgtatgtaatcgatttttcgtgagagtgaagcctgatcagggttagccgat

SEQ ID NO: 44 - DNA, 23 bp, Artificial Sequence
  primer b1867A-F
tgcgctggtggtgatcgatttac

SEQ ID NO: 45 - DNA, 44 bp, Artificial Sequence
  primer b1867A-R
tggcacagggcaacagggtaaacgcgttgaggatctcttccacg

SEQ ID NO: 46 - DNA, 21 bp, Artificial Sequence
  primer argS-F
ttaccctgttgccctgtgcca

SEQ ID NO: 47 - DNA, 51 bp, Artificial Sequence
  primer argS-R
gcgatgtgatattgctctcctatggagatcggctaaccctgatcaggcttc

SEQ ID NO: 48 - DNA, 27 bp, Artificial Sequence
  primer b1901C-F
ctccataggagagcaatatcacatcgc

SEQ ID NO: 49 - DNA, 54 bp, Artificial Sequence
  primer b1901C-R
gccgatcaacgtctcattttcgccaaaaatgtgatcgtggagtcaattctgacg

SEQ ID NO: 50 - DNA, 47 bp, Artificial Sequence
  primer b1900B-F
ggctgatcagctagcccatgggtatgaccagcattgagttggcagcg

SEQ ID NO: 51 - DNA, 22 bp, Artificial Sequence
  primer b1900B-R
atgtcacaatccgctatggcgg

SEQ ID NO: 52 - DNA, 18 bp, Artificial Sequence
  I-SceI restriction enzyme site
tagggataacagggtaat

SEQ ID NO: 53 - DNA, 1429 bp, Artificial Sequence
  sacB gene
ttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcat

SEQ ID NO: 54 - DNA, 3535 bp, Artificial Sequence
  DNA fragment of b0004˜b0024 deletion
ccgccgattttgctgcgttgcgtaaattgatgatgaatcatcagtaaaatccagacatgaagctgatacgcggcatacataatctcagccaggccccgcaagaagggtgtgtgctgactattggtaatttcgacggcgtgcatcgcggtcatcgcgcgctgttacagggcttgcaggaagaagggcgcaagcgcaacttaccggtgatggtgatgctttttgaacctcaaccactggaactgtttgctaccgataaagccccggcaagactgacccggctgcgggaaaaactgcgttaccttgcagagtgtggcgttgattacgtgctgtgcgtgcgtttcgacaggcgtttcgcggcgttaaccgcgcaaaatttcatcagcgatcttctggtgaagcatttgcgcgtaaaatttcttgccgtaggtgatgatttccgctttggcgctggtcgtgaaggcgatttcttgttattacagaaagctggcatggaatacggcttcgatatcaccagtacgcaaactttttgcgaaggtggcgtgcgcatcagcagcaccgccgtgcgtcaggcccttgcggatgacaatctgtacgactcactatagggagaccggaattcgagctcggtaccttttggcgaaaatgagacgttgatcggcacgtaagaggttccagctttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccgaaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcctcactgattaagcattggtaactgtcagatccggggaatttatgggattcacctttatgttgataagaaataaaagaaaatgccaataggatatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatccgctagggataacagggtaatatagatcctctagagtcgacctgcagtcggtactcaatcgccggttaaccttgaccgctgtacaaggtatactcgg

SEQ ID NO: 55 - DNA, 4543 bp, Artificial Sequence
  DNA fragment of b0980-b1052 deletion
actgttggtgtgattcatctgcgttcacagctggcgaccaaacgcgctgcactgttggtgatgctgtgctttttgctggcgggttactggctgtgggtcggtattgatggctttgtactgctcgcccaggatgctaacggtccttccaatccgttaatgaaactggtggcagtgctacctggtgcctggatgaataattttgtcgagtcgcccgttttgtggatcttcccgctgctgggattcttctgcccattgctgacggtgatggcgatttatcgtggtcgcccgggttggggatttttgatggcatcattgatgcaatttggcgtgattttcacggcaggcatcacgctgttcccctttgtcatgccgtcaagcgtgagtccgatctccagcctgacgttgtgggacagtacttccagtcagctgacgctgagcattatgttggtaatcgtgctgatatttttgcccattgtgttgctctacactctctggagctactacaaaatgtgggggcgcatgacaacagaaactctccgccgtaacgaaaacgagttgtactaaggagcagaaacaatgtggtaatcagttcgatacctggggtattcgacgacgacgtagactgttccatgaatagactgcgttgaataacactacgccagcggtgacgagaaataccgctctgaaaccgtagttcgctgaaatcgctgctcccatcaatggtccggtaacgttgccaatatcacgaaacgattggttatagctgaagatacgcccggcgatctggttgctcgagttgtaaaccaacagtgtctgtacggcggggagtagtgcaccatcggcggcaccgagcaaaaaacgtaaaatcccaagttgcaatggcgtctgaacgtaagacattgggatcaacagcagtacagaaaagatcagcgctgtaatcaggatcttttcgggtccgattcgatcgccaagtttgccgagtcgtggtgcacttagcagagccgccacgcctggcaccgaggcgatcatgccactgataaaggcgacgttactgacgttacccgccagttcgcggacatacagcgtcagaatttttggcgaaaatgagacgttgatcggcacgtaagaggttccagctttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccgaaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcctcactgattaagcattggtaactgtcagatccggggaatttatgggattcacctttatgttgataagaaataaaagaaaatgccaataggatatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatccgctagggataacagggtaatatagatcctctagagtcgacctgcaggcatgcaagcttggcactggctgatcagctagcccatgggtatgagcattattagtcgcactataccggggcaggggcatggcaaccaatattatcccggtgtgcaatgggatgtgcgtgactccgcctggcgttatgtcacgatccttgatctgccattctctctggtttttgatactttactgctgccgatcgacattcatcatggcccgtatgagtgattaacgttcatcccactcatcagctgctgcgcgtccttcctcggtatccagcggtggctcaagctgaaattccccctcgtcccattcatgtaatgtattctcttcctgccactcctggcgtatctctatctcatcatagtcgccatcaaaaacactttgcgcggcttcaccgctaagcataggtaaacattcaccttcttccccttcgtcggcaaaaaactcaacttgccacatgatgtcgccgtcctgcaaaacgtatttttgggcattgaactgttgcacattcgcatcttcggcgtcgatgccggggttgtctgcaagaaattcttcgcgtgcagcgtcaatggcttcttcaagcgttgcgtacatggtcatc

SEQ ID NO: 56 - DNA, 4597 bp, Artificial Sequence
  DNA fragment of b1137-b1158 deletion
ctgatggatggcgctaaactgctgaaatgttcagagtttggtgacgcgatcatcgaaaacatgtaatgccgtagtttgttaaatttattaacgggagcgtaacgctcccgttgttttttgttaggctgctaacggttatcaaaattttatcaaaaaaagttatcaaaacccctcggtagttttggggtaggctggccggtcaggtggtagttctactactagtctcccacatagatattccttagctttttattattgctggcggacgctcgttaatatttaaggtcttcattgattaagacatccccaaagttagttatgtattcactgttattaggacaattatgaattaccactccttacacccgctcaaatattgttaaattgccggttttgtatcaactactcacccgggactcgccaggggacagccaacaggcattgggtgcaatcaccttagcgttcaggtacatgcggaatgtaaaaaaggccgcgagcgcggccccttcacatacatcttcaacgcctgaagagatcctgcgacgggcaataaataatggggagatcgtccctttttaccaacctgtggtaaatggtcgggaagggacattgcggggagttgaggtgttagcccgctggaaacaacctcacggtggatatatatcacccgcggcatttattccacttgctgaaaaatccggattaatcgttccgcttacgcaaagcctgatgaatcaggttgccagacagatgaacgctatcgcgagtaaattgccggaaggttttcatattggaattaattttagcgcttcgcatattatttcgccgacgtttgtcgacgagtgtttaaatttccgtgacagttttacccgccgcgatttaaaccttgttctggaagtcaccgagcgtgagccattgaatgttgatgaaagtctggttcagcggttgaacatactgcatgaaaatggttttgtcatcgcactggatgatttcggtactggctactcagggctttcttatcttcatgacctgcatattgattatatcaaaattgatcacagtttcgttggccgcgtaaacgcagacccagaatcaacccgaatttttggcgaaaatgagacgttgatcggcacgtaagaggttccagctttcaccataatgaaataagatcactaccgggcgtattttttgagttatcgagattttcaggagctaaggaagctaaaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatttttttaaggcagttattggtgcctcactgattaagcattggtaactgtcagatccggggaatttatgggattcacctttatgttgataagaaataaaagaaaatgccaataggatatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatccgctagggataacagggtaatatagatcctctagagtcgacctgcaggcatgcaagcttggcactggctgatcagctagcccatgggtatgatccagctctggtattccgcaaaagcagagtacctggcgggagcgagatatgccgccaacaatatcaatcatatacttgaagaagcgtcacaagcgactcaaacagcggttaacattgccgggaaggaatgcaacctcgaggagcaatatcagcttggcactgaagcagctctgaaacctcacctgcgcacaatcatcattctcaaacagggaatagtctggtgtacatccctgcctgggaatcgggtcctgttgtctcgtattcctgttttcccggacagtaatttactgttggctccagcaatcgacaccgttaatagattacctatcctgctctatcagaaccaatttgcagatacgcgcattttggttacgataagcgatcagcatattcgtggggcacttaatgtacccttgaaaggggtaaggtatgtattacgcgtggcggatgacattattgg

SEQ ID NO: 57 - DNA, 6549 bp, Artificial Sequence
  DNA fragment of b1867-b1901 deletion
tgcgctggtggtgatcgatttacaagaaggcatcttaccttttgccggtggtccacatactgccgatgaggtagttaatcgcgccgggaagctggcggcgaaatttcgcgccagcggtcagcccgtgtttctggtgcgcgttggctggtctgccgattacgctgaagcattaaaacagccggtcgatgcgccctcaccggctaaagtgttgcccgaaaattggtggcaacatcctgctgcattaggtgcaaccgacagcgatatcgaaatcatcaaacgtcaatggggtgcgttttacggtacggatctggagttgcaattacgccgccgaggtatcgatacaatagtgttatgtgggatctcgaccaatatcggtgttgaatccaccgcccgcaatgcctgggaacttggctttaatctggtgattgccgaagacgcctgtagtgccgccagcgccgagcagcacaataacagtatcaaccatatctacccgcgcatcgcccgtgtgcgtagcgtggaagagatcctcaacgcgtttaccctgttgccctgtgccaaccaaccgctgatttcacgccgcttctgatgcaatagtgaaaacggcaatacgccacgcgcacgttgctgacgaaaacagccatttgcagtatactcccgccctaatttctttaactggtgcgggcaatttttgctcgcttcatcaatgtaaggtattccggtgaatattcaggctcttctctcagaaaaagtccgtcaggccatgattgcggcaggcgcgcctgcggattgcgaaccgcaggttcgtcagtcagcaaaagttcagttcggcgactatcaggctaacggcatgatggcagttgctaaaaaactgggtatggcaccgcgacaattagcagagcaggtgctgactcatctggatcttaacggtatcgccagcaaagttgagatcgccggtccaggctttatcaacattttccttgatccggcattcctggctgaacatgttcagcaggcgctggcgtccgatcgtctcggtgttgctacgccagaaaaacagaccattgtggttgactactctgcgccaaacgtggcgaaagagatgcatgtcggtcacctgcgctctaccattattggtgacgcagcagtgcgtactctggagttcctcggtcacaaagtgattcgcgcaaaccacgtcggcgactggggcactcagttcggtatgctgattgcatggctggaaaagcagcagcaggaaaacgccggtgaaatggagctggctgaccttgaaggtttctaccgcgatgcgaaaaagcattacgatgaagatgaagagttcgccgagcgcgcacgtaactacgtggtaaaactgcaaagcggtgacgaatatttccgcgagatgtggcgcaaactggtcgacatcaccatgacgcagaaccagatcacctacgatcgtctcaacgtgacgctgacccgtgatgacgtgatgggcgaaagcctctacaacccgatgctgccaggaattgtggcggatctcaaagccaaaggtctggcagtagaaagcgaaggggcgaccgtcgtattccttgatgagtttaaaaacaaggaaggcgaaccgatgggcgtgatcattcagaagaaagatggcggctatctctacaccaccactgatatcgcctgtgcgaaatatcgttatgaaacactgcatgccgatcgcgtgctgtattacatcgactcccgtcagcatcaacacctgatgcaggcatgggcgatcgtccgtaaagcaggctatgtaccggaatccgtaccgctggaacaccacatgttcggcatgatgctgggtaaagacggcaaaccgttcaaaacccgcgcgggtggtacagtgaaactggccgatctgctggatgaagccctggaacgtgcacgccgtctggtggcagaaaagaacccggatatgccagccgacgagctggaaaaactggctaacgcggttggtattggtgcggtgaaatatgcggatctctccaaaaaccgcaccacggactacatcttcgactgggacaacatgctggcgtttgagggtaataccgcgccatacatgcagtatgcatacacgcgtgtattgtccgtgttccgtaaagcagaaattgacgaagagcaactggctgcagctccggttatcatccgtgaagatcgtgaagcgcaactggcagctcgcctgctgcagtttgaagaaaccctcaccgtggttgcccgtgaaggcacgccgcatgtaatgtgtgcttacctgtacgatctggccggtctgttctctggcttctacgagcactgcccgatcctcagcgcagaaaacgaagaagtgcgtaacagccgtctaaaactggcacaactgacggcgaagacgctgaagctgggtctggatacgctgggtattgagactgtagagcgtatgtaatcgatttttcgtgagagtgaagcctgatcagggttagccgatctccataggagagcaatatcacatcgcagaattacagtgagaacgtgcataaatttagcgggaaaagacataagggaaagccaatttgtcagacaaattgtcgaatgcacagcagattaatccataagattagcctggaaatccttgttgtctttggtacccatgcgggatgtcttctttttaaccagtcaataggccgcattacctggcgttgagtttttgaaatggtgtaataaccgcaactcaaagatgtggaaaatgcacgtcattcatttcgtcattaattatcactgtgctcattaattaacagaacacgtataatgagagccatctcgcaaaaatgaaaaaacgttttataaaatcatcacttcatcatgaattcaaattcattgattaatatcaacaagatacaaaaagcactatcattaaaattcattgcagttacattgatttcatcaatgaaatgtaaaaatatataaacttgatgatttaagcattttcttatacccgttcagacgttattcttatttcagatcatcgtcagaattgactccacgatcacatttttggcgaaaatgagacgttgatcggcacgtaagaggttccagctttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccgaaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcctcactgattaagcattggtaactgtcagatccggggaatttatgggattcacctttatgttgataagaaataaaagaaaatgccaataggatatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatccgctagggataacagggtaatatagatcctctagagtcgacctgcaggcatgcaagcttggcactggctgatcagctagcccatgggtatgaccagcattgagttggcagcgtcaaatgcccccgggatgtcgttagatttggtaggtacctgataaatttgtttttccgggaatccggccgctttcagcgcatccatagatcccgtagtacggcggcgggcggtatccagttcgttggcggtaatcgccatcaccgcgctttctttgacatcccagccacgtttctgcatctctttatacagttcctggccctgacgttcgccaattttagtcgccgccatcatcaccagcggaacggtatccattggcttacctttggcgttaacaaactggtcatccacggcaatgactttcatatcgtagccacgcgctttcgcgacgatggcagagccgagtttggggtccggagtacaaataacgaaaccttttgcgccactggcagccaggctgtcgatcgcgttcaatgttttttcgccatccggcacggcaatcttaataacctcaaaccctaaatccttcccggctttatcggcaaacttccattcggtctggaaccacggctcttccggttgcttcaccagaaaaccgagcttcaggttctccgccatagcggattgtgacat